(19) United States
(12) Patent Application Publication
(10) Pub. No.: US 2005/0139123 A1
(43) Pub. Date: Jun. 30, 2005

(54) Azo dye, colored composition for image formation, ink, method of ink-jet recording, heat-sensitive recording material, color toner and color filter

(75) Inventor: Toshiki Fujiwara, Minami-Ashigara-shi (JP)

Correspondence Address: BURNS DOANE SWECKER & MATHIS L L P, POST OFFICE BOX 1404, ALEXANDRIA, VA, 22313-1404, US

(73) Assignee: Fuji Photo Film Co., Ltd., Minami-Ashigara-shi (JP)

(21) Appl. No.: 10/984,857

(22) Filed: Nov. 10, 2004

(30) Foreign Application Priority Data
Dec. 26, 2003 (JP) 2003-434595

(51) Int. Cl. C09D 11/00; G02B 5/22; C07D 417/02; C07D 498/02

(52) U.S. Cl. 106/31.48; 106/31.52; 106/31.5; 534/726; 534/744; 359/885

(57) ABSTRACT

A colored composition for image formation which comprises an azo dye represented by formula (1); an ink for ink-jet recording and a method of ink-jet recording each comprising or using the composition; and a heat-sensitive recording material, color toner, and color filter each formed from the composition.

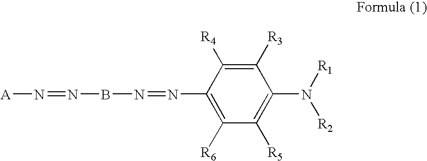

In the formula, R₁ represents a substituted or unsubstituted, aryl or heterocyclic group; R₂ represents a hydrogen atom, a substituted or unsubstituted, alkyl, aryl, or heterocyclic group, etc.; and R₃, R₄, R₅, and R₆ each independently represents a hydrogen atom, a halogen atom, an alkyl group, an aromatic group, an alkylsulfonylamino group or an acylamino group, etc.

### BACKGROUND OF THE INVENTION

1. Field of the Invention

The present invention relates to an aromatic nitrogen-containing heterocyclic azo dye and a colored composition for use in, e.g., image formation which contains the azo dye. The invention further relates to an ink, method of ink-jet recording, heat-sensitive recording material, color toner, and color filter which contain or use the dye.

2. Description of the Related Art

Image-recording materials which are mainly used in recent years are materials especially for forming color images. Specifically, the recording materials in extensive use include recording materials for ink-jet recording, recording materials for thermal transfer recording, recording materials for electrophotography, silver halide photosensitive materials for transfer printing, printing inks, and recording pens. Furthermore, color filters for recording/reproducing color images are used in the photographing elements, e.g., CCDs, of cameras and in displays such as LCDs and PDPs.

In these color image-recording materials and color filters, colorants (dyes or pigments) of the three primary colors according to the so-called additive color-mixing method or subtractive color-mixing method are used in order to reproduce or record full-color images. Virtually, however, there are no fast colorants which have absorption characteristics capable of realizing a preferred color reproduction region and can withstand various use conditions and environmental conditions. Improvements in such points are strongly desired.

The colorants for use in those applications are required to have the following and other properties common to these: to have absorption characteristics preferred for color reproduction; to be satisfactory in fastness to environmental conditions under which the colorants are to be used, such as light resistant, heat resistance, moisture resistance, resistance to oxidizing gases including ozone, and fastness to chemicals, e.g., sulfurous acid gas; and to have a large molar extinction coefficient.

Known as a technique effective in enhancing fastness to oxidizing gases including ozone is a monoazo magenta dye including an aniline coupler in which at least one of the substituents on the nitrogen atom of the aniline moiety is an aryl substituent (see JP-A-O₂-053777). However, no dyes having enhanced fastness to oxidizing gases are known with respect to disazo and polyazo dyes.

### SUMMARY OF THE INVENTION

The invention is intended to eliminate the above-described problems of related-art techniques and accomplish the following objects.

Namely, an object of the invention is to provide a novel azo dye excellent in hue and fastness.

Another object of the invention is to provide a colored composition for image formation which gives a colored image or colored material excellent in hue and fastness and is advantageously usable in preparing an ink for printing such as ink-jet printing, ink sheet for use in heat-sensitive recording materials, color toner for electrophotography, color filter for use in displays such as LCDs and PDPs or photographing elements such as CCDs, dyeing solution for dyeing various fibers, etc.

Still another object of the invention is to provide an ink which has a satisfactory hue and can form images having high fastness to light and active gases in the environment, in particular ozone gas, and to provide a method of ink-jet recording.

A further object of the invention is to provide a heat-sensitive recording material which gives images having excellent light fastness and a bright hue.

Still a further object of the invention is to provide a color toner which has excellent light fastness and shows faithful color reproduction and high OHP quality.

Still a further object of the invention is to provide a color filter excellent in color reproduction and light fastness.

The present inventors made close investigations on various dye compound derivatives in order to develop a dye having a satisfactory hue and high fastness to light and ozone. As a result, it has been found that the problems described above can be eliminated by the azo dye of the following general formula (1).

Namely, the invention provides an azo dye, a colored composition for image formation containing the azo dye, an ink, a method of ink-jet recording, a heat-sensitive recording material, a color toner, and a color filter which respectively have the following constitutions. Those objects of the invention are thus accomplished.

(1) An azo dye represented by formula (1): 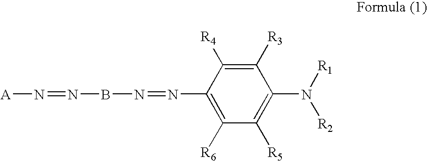
wherein A and B each independently represents an aryl group or a heterocyclic group;
R₁ represents a substituted or unsubstituted aryl group; or a substituted or unsubstituted heterocyclic group;
R₂ represents a hydrogen atom; a substituted or unsubstituted alkyl group; a substituted or unsubstituted aryl group; a substituted or unsubstituted cycloalkyl group; a substituted or unsubstituted alkenyl group; a substituted or unsubstituted aralkyl group; a substituted or unsubstituted heterocyclic group; a substituted or unsubstituted acyl group; an alkyl-substituted sulfonyl group; or an aryl-substituted sulfonyl group;
R₃, R₄, R₅ and R₆ each independently represents a hydrogen atom; a halogen atom; an aliphatic group; an aromatic group; a heterocyclic group; a cyano group; a carboxyl group; a carbamoyl group; an alkoxycarbonyl group; an aryloxycarbonyl group; a heterocycle-oxycarbonyl group; an acyl group; a hydroxy group; an alkoxy group; an aryloxy group; a heterocycle-oxy group; a silyloxy group; an acyloxy group; a carbamoyloxy group; an alkoxycarbonyloxy group; an aryloxycarbonyloxy group; an amino group substituted with an alkyl group, an aryl group or a heterocyclic group; an acylamino group; an ureido group; a sulfamoylamino group; an alkoxycarbonylamino group; an aryloxycarbonylamino group; an alkylsulfonylamino group; an arylsulfonylamino group; a heterocycle-sulfonylamino group; a nitro group; an alkylthio group; an arylthio group; a heterocycle-thio group; an alkylsulfonyl group; an arylsulfonyl group; a heterocycle-sulfonyl group; an alkylsulfinyl group; an arylsulfinyl group; a heterocycle-sulfinyl group; a sulfamoyl group; or a sulfo group;
A, B, R₁, R₂, R₃, R₄, R₅ and R₆ may be substituted; and

R₁ and R₂ may be bonded to R₃ and R₅, respectively, to form a ring.

(2) The azo dye as described in (1) above,
wherein A is a benzene ring which may be substituted, or a naphthalene ring which may be substituted; and B is a benzene ring which may be substituted a naphthalene ring which may be substituted, or a heterocycle selected from (a), (b), (c), (d) or (e): 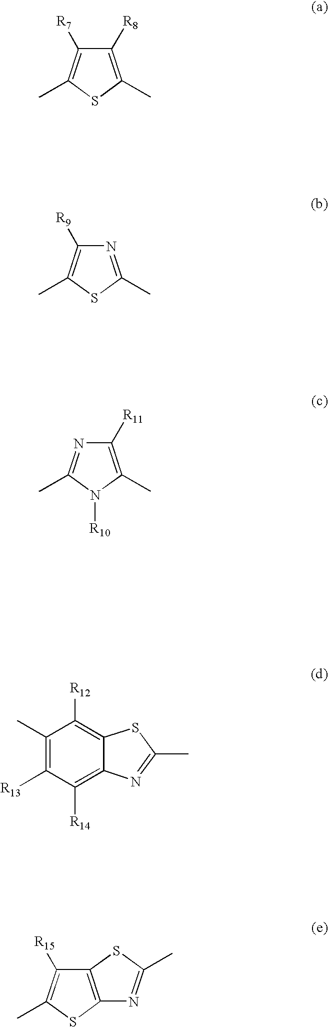
wherein R₇ to R₁₅ each independently represents a hydrogen atom; a halogen atom; an aliphatic group; an aromatic group; a heterocyclic group; a cyano group; a carboxyl group; a carbamoyl group; an alkoxycarbonyl group; an aryloxycarbonyl group; a heterocycle-oxycarbonyl group; an acyl group; a hydroxy group; an alkoxy group; an aryloxy group; a heterocycle-oxy group; a silyloxy group; an acyloxy group; a carbamoyloxy group; an alkoxycarbonyloxy group; an aryloxycarbonyloxy group; an amino group substituted with an alkyl group, an aryl group or a heterocyclic group; an acylamino group; an ureido group; a sulfamoylamino group; an alkoxycarbonylamino group; an aryloxycarbonylamino group; an alkylsulfonylamino group; an arylsulfonylamino group; a heterocycle-sulfonylamino group; a nitro group; an alkylthio group; an arylthio group; a heterocycle-thio group; an alkyl sulfonyl group; an arylsulfonyl group; a heterocycle-sulfonyl group; an alkylsulfinyl group; an arylsulfinyl group; a heterocycle-sulfinyl group; a sulfamoyl group; or a sulfo group; and
R₇ to R₁₅ may be substituted.

(3) A colored composition for image formation comprising at least one azo dye represented by formula (1) described in (1) above.

(4) An ink comprising at least one azo dye represented by formula (1) described in (1) above.

(5) A method of ink-jet recording comprising forming an image with an ink as described in (4) above.

(6) A heat-sensitive recording material comprising an azo dye as described in (1) or (2) above.

(7) A color toner comprising an azo dye as described in (1) or (2) above.

(8) A color filter comprising an azo dye as described in (1) or (2) above.

### DETAILED DESCRIPTION OF THE INVENTION

Azo Dye

First, the azo dye represented by general formula (1) in the invention (often referred to as “dye of the invention”) is explained in detail.

In general formula (1), A and B each independently represents an aromatic group which may be substituted or a heterocyclic group which may be substituted. Examples of the aromatic group include a benzene ring and a naphthalene ring, and these rings may be substituted with any substituent(s). Examples of such substituents include the substituents represented by R₃, R₄, R₅, and R₆.

Examples of the heteroatom(s) of the heterocycle include nitrogen, oxygen, and sulfur. The heterocycle may have been fused with an aliphatic ring or aromatic ring or with another heterocycle.

Symbol A preferably is a benzene ring which may be substituted, or a naphthalene ring which may be substituted. Preferred examples of B include a heterocycle shown below besides a benzene ring which may be substituted and a naphthalene ring which may be substituted.

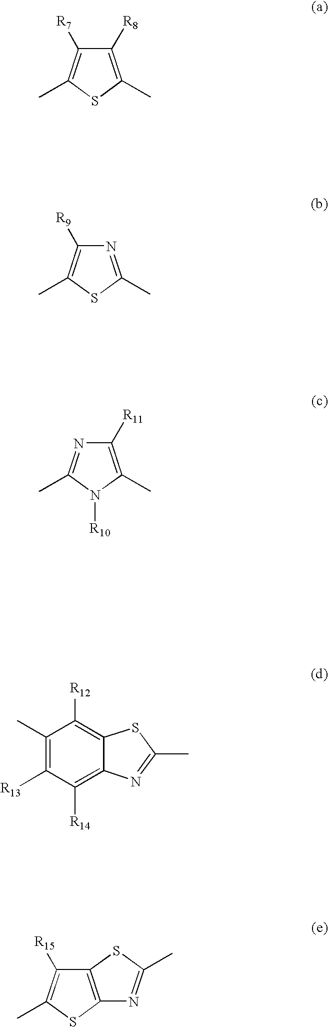

In general formulae (a) to (e), R₇ to R₁₅ represent the same substituents as substituents R₃, R₄, R₅, and R₆, which will be explained later.

Preferred of general formulae (a) to (e) are the thiophene ring, thiazole ring, imidazole ring, and thienothiazole ring represented by general formulae (a), (b), (c), and (e). Especially preferred is the case where A is a naphthalene ring and B is general formula (a) or (b).

R₃, R₄, R₅ and R₆ each independently represents a hydrogen atom; a halogen atom; an aliphatic group; an aromatic group; a heterocyclic group; a cyano group; a carboxyl group; a carbamoyl group; an alkoxycarbonyl group; an aryloxycarbonyl group; a heterocycle-oxycarbonyl group; an acyl group; a hydroxy group; an alkoxy group; an aryloxy group; a silyloxy group; an acyloxy group; a carbamoyloxy group; a heterocycle-oxy group; an alkoxycarbonyloxy group; an aryloxycarbonyloxy group; an amino group substituted with an alkyl group, an aryl group or a heterocyclic group; an acylamino group; an ureido group; a sulfamoylamino group; an alkoxycarbonylamino group; an aryloxycarbonylamino group; an alkylsulfonylamino group; an arylsulfonylamino group; a heterocycle-sulfonylamino group; a nitro group; an alkylthio group; an arylthio group; a heterocycle-thio group; an alkylsulfonyl group; an arylsulfonyl group; a heterocycle-sulfonyl group; an alkylsulfinyl group; an arylsulfinyl group; a heterocycle-sulfinyl group; a sulfamoyl group; or a sulfo group, provided that each group may have been further substituted.

Examples of the aromatic ring represented by A include a benzene ring and a naphthalene ring, and these rings may have been substituted with any substituent(s). These substituents preferably are electron-attracting groups. Specifically, electron-attracting groups having a Hammett's σp value of 0.2 or higher are preferred.

An explanation is given here on the Hammett's substituent constant σp value.

Hammett's rule is an empirical rule proposed by L. P. Hammett in 1935 in order to quantitatively discuss the influences of substituents of benzene derivatives on reactions of the derivatives or equilibrium thereof. At present, this rule is extensively regarded as valid. The substituent constants required of Hammett's rule include σp and σm, and values of these can be found in many general books. For example, these substituent constants are described in detail in J. A. Dean, ed., Lange's Handbook of Chemistry, 12th edition, 1979 (Mc Graw-Hill) and Kagaku-No Ry\overline{o}iki, extra edition, No. 122, pp. 96-103, 1979 (Nanko-do). In this invention, substituents are limited or explained in terms of their Hammett's substituent constants σp. However, this does not mean that the substituents in the invention are limited to the substituents which have known values of σp found in these books, and it is a matter of course that the substituents in the invention include substituents which each have an unknown value of op but may give a value in that range when examined in accordance with Hammett's rule. Although general formula (1) in the invention includes compounds which are not benzene derivatives, values of σp are used as a measure of the electronic effect of the substituents regardless of substitution position. In the invention, values of σp are used in such a sense.

Examples of electron-attracting groups having a value of Hammett's substituent constant σp of 0.60 or higher include cyano, nitro, alkylsulfonyl groups (e.g., methanesulfonyl), and arylsulfonyl groups (e.g., benzenesulfonyl).

Examples of electron-attracting groups having a Hammett op value of 0.45 or higher include acyl groups (e.g., acetyl), alkoxycarbonyl groups (e.g., dodecyloxycarbonyl), aryloxycarbonyl groups (e.g., m-chlorophenoxycarbonyl), alkylsulfinyl groups (e.g., n-propylsulfinyl), arylsulfinyl groups (e.g., phenylsulfinyl), sulfamoyl groups (e.g., N-ethylsulfamoyl and N,N-dimethylsulfamoyl), and halogenoalkyl groups (e.g., trifluoromethyl) besides the electron-attracting groups shown above.

Examples of electron-attracting groups having a value of Hammett's substituent constant σp of 0.30 or higher include acyloxy groups (e.g., acetoxy), carbamoyl groups (e.g., N-ethylcarbamoyl and N,N-dibutylcarbamoyl), halogenoalkoxy groups (e.g., trifluoromethyloxy), halogenoaryloxy groups (e.g., pentafluorophenyloxy), sulfonyloxy groups (e.g., methylsulfonyloxy), halogenoalkylthio groups (e.g., difluoromethylthio), aryl groups substituted with two or more electron-attracting groups each having a σp value of 0.15 or higher (e.g., 2,4-dinitrophenyl and pentachlorophenyl), and heterocylces (e.g., 2-benzoxazolyl, 2-benzothiazolyl, and 1-phenyl-2-benzimidazolyl) besides the electron-attracting groups enumerated above.

Examples of electron-attracting groups having a σp value of 0.20 or higher include halogen atoms besides the electron-attracting groups enumerated above.

Preferred examples of the substituents represented by R₃, R₄, R₅, and R₆ include a hydrogen atom; a halogen atom; a cyano group; an aliphatic group; an aromatic group; a hydroxy group; an alkoxy group; an amino group substituted with an alkyl group, an aryl group or a heterocyclic group; an acylamino group; an ureido group; an alkylsulfonylamino group; an arylsulfonylamino group; an alkylthio group; an arylthio group ¥; and a heterocycle-thio group. Preferred of these are a hydrogen atom; a halogen atom; a cyano group; an aliphatic group; an aromatic group; an acylamino group; an alkylsulfonylamino group; an arylsulfonylamino group; and an amino group substituted with an alkyl group, an aryl group or a heterocyclic group.

R₁ represents a substituted or unsubstituted aryl group; or a substituted or unsubstituted heterocyclic group; and R₂ represents a hydrogen atom; a substituted or unsubstituted alkyl group; a substituted or unsubstituted aryl group; a substituted or unsubstituted cycloalkyl group; a substituted or unsubstituted alkenyl group; a substituted or unsubstituted aralkyl group; a substituted or unsubstituted heterocyclic group; a substituted or unsubstituted acyl group; or an alkyl- or aryl-substituted sulfonyl group. These groups may have been substituted with any substituent (s). Examples of such substituents include the substituents represented by R₃, R₄, R₅, and R₆.

R₁ preferably is a substituted phenyl group, and R₂ preferably is a hydrogen atom, substituted or unsubstituted aryl group, substituted or unsubstituted cycloalkyl group, substituted or unsubstituted heterocyclic group, acyl group, or alkyl-or aryl-substituted sulfonyl group. More preferably, R₂ is a hydrogen atom, substituted or unsubstituted aryl group, substituted or unsubstituted heterocyclic group, acyl group, or alkyl- or aryl-substituted sulfonyl group.

In the case where the azo dye represented by general formula (1) is a water-soluble dye, the dye preferably further has one or more ionic hydrophilic groups as substituents in any positions in A, B, R₁, R₂, R₃, R₄, R₅, and R₆. Examples of the ionic hydrophilic groups as substituents include sulfo, phosphono, carboxyl, and quaternary ammonium groups. Of these ionic hydrophilic groups, carboxyl and sulfo are preferred, and sulfo is especially preferred. The carboxyl, phosphono, and sulfo groups may be in the state of a salt. Examples of the counter ion as a component of the salt include an ammonium ion, alkali metal ions (e.g., lithium ion, sodium ion, and potassium ion), and organic cations (e.g., tetramethyl guanidinium ion and tetramethylammonium ion).

The substituents on the aryl groups and heterocyclic groups represented by R₁ and R₂ and the substituents represented by R₃, R₄, R₅, and R₆ will be explained below in detail.

Examples of the halogen atom include fluorine, chlorine, and bromine atoms.

The term aliphatic group as used in this specification means any of alkyl groups, substituted alkyl groups (including aralkyl groups and substituted aralkyl groups), alkenyl groups, substituted alkenyl groups, alkynyl groups, and substituted alkynyl groups.

The aliphatic group may have one or more branches and may be in a ring form. The number of carbon atoms of the aliphatic group is preferably 1 to 20, more preferably 1 to 16. In each of the aralkyl groups and substituted aralkyl groups, the aryl moiety preferably is phenyl or naphthyl, and especially preferably is phenyl. Examples of the aliphatic group include methyl, ethyl, butyl, isopropyl, t-butyl, hydroxyethyl, methoxyethyl, cyanoethyl, trifluoromethyl, 3-sulfopropyl, 4-sulfobutyl, cyclohexyl, benzyl, 2-phenethyl, vinyl, and allyl.

The term aromatic group as used in this specification means an aryl group or a substituted aryl group. The aryl group preferably is phenyl or naphthyl, and especially preferably is phenyl. The number of carbon atoms of the aromatic group is preferably 6 to 20, more preferably 6 to 16.

Examples of the aromatic group include phenyl, p-tolyl, p-methoxyphenyl, o-chlorophenyl, and m-(3-sulfopropylamino)phenyl.

The heterocyclic group may be a heterocyclic group having one or more substituents or an unsubstituted heterocyclic group. The heterocycle may have been fused with an aliphatic ring or aromatic ring or with another heterocycle. The heterocyclic group preferably is a five- or six-membered heterocyclic group. Examples of the substituents include aliphatic groups, halogen atoms, alkylsulfonyl groups, arylsulfonyl groups, acyl groups, acylamino groups, sulfamoyl, carbamoyl groups, and ionic hydrophilic groups. Examples of the heterocyclic group include a 2-pyridyl, 2-thienyl, 2-thiazolyl, 2-benzothiazolyl, 2-benzoxazolyl, and 2-furyl.

The carbamoyl group may be a carbamoyl group having one or more substituents or unsubstituted carbamoyl. Examples of the substituents include alkyl groups. Examples of the carbamoyl group include methylcarbamoyl and dimethylcarbamoyl.

The alkoxycarbonyl group may be an alkoxycarbonyl group having one or more substituents or an unsubstituted alkoxycarbonyl group. The alkoxycarbonyl group preferably is an alkoxy carbonyl group having 2 to 12 carbon atoms. Examples of the substituents include ionic hydrophilic groups. Examples of the alkoxycarbonyl group include methoxycarbonyl and ethoxycarbonyl.

The aryloxycarbonyl group may be an aryloxycarbonyl group having one or more substituents or an unsubstituted aryloxycarbonyl group. The aryloxycarbonyl group preferably is an aryloxy carbonyl group having 7 to 12 carbon atoms. Examples of the substituents include ionic hydrophilic groups. Examples of the aryloxycarbonyl group include phenoxycarbonyl.

The acyl group may be an acyl group having one or more substituents or an unsubstituted acyl group. The acyl group preferably is an acyl group having 1 to 12 carbon atoms. Examples of the substituents include ionic hydrophilic groups. Examples of the acyl group include acetyl and benzoyl.

The alkoxy group may be an alkoxy group having one or more substituents or an unsubstituted alkoxy group. The alkoxy group preferably is an alkoxy group having 1 to 12 carbon atoms. Examples of the substituents include alkoxy groups, hydroxyl, and ionic hydrophilic groups. Examples of the alkoxy group include methoxy, ethoxy, isopropoxy, methoxyethoxy, hydroxyethoxy, and 3-carboxypropoxy.

The aryloxy group may be an aryloxy group having one or more substituents or an unsubstituted aryloxy group. The aryloxy group preferably is an aryloxy group having 6 to 12 carbon atoms. Examples of the substituents include alkoxy groups and ionic hydrophilic groups. Examples of the aryloxy group include phenoxy, p-methoxyphenoxy, and o-methoxyphenoxy.

The acyloxy group may be an acyloxy group having one or more substituents or an unsubstituted acyloxy group. The acyloxy group preferably is an acyloxy group having 1 to 12 carbon atoms. Examples of the substituents include ionic hydrophilic groups. Examples of the acyloxy group include acetoxy and benzoyloxy.

The carbamoyloxy group may be a carbamoyloxy group having one or more substituents or unsubstituted carbamoyloxy. Examples of the substituents include alkyl groups. Examples of the carbamoyloxy group include N-methylcarbamoyloxy.

The heterocycle-oxy group may be a heterocycle-oxy group having one or more substituents or an unsubstituted heterocycle-oxy group. The heterocycle-oxy group preferably is a heterocycle-oxy group having 2 to 20 carbon atoms. Examples of the substituents include alkyl groups, alkoxy groups, and ionic hydrophilic groups. Examples of the heterocycle-oxy group include 3-pyridyloxy and 3-thienyloxy.

The silyloxy group preferably is a silyloxy group substituted with one or more aliphatic or aromatic groups having 1 to 20 carbon atoms. Examples of the silyloxy group include trimethylsilyloxy and diphenylmethylsilyloxy.

The alkoxycarbonyloxy group may be an alkoxycarbonyloxy group having one or more substituents or an unsubstituted alkoxycarbonyloxy group. The alkoxycarbonyloxy group preferably is an alkoxycarbonyloxy group having 2 to 20 carbon atoms. Examples of the alkoxycarbonyloxy group include methoxycarbonyloxy and isopropoxycarbonyloxy.

The aryloxycarbonyloxy group may be an aryloxycarbonyloxy group having one or more substituents or an unsubstituted aryloxycarbonyloxy group. The aryloxycarbonyloxy group preferably is an aryloxycarbonyloxy group having 7 to 20 carbon atoms. Examples of the aryloxycarbonyloxy group include phenoxycarbonyloxy.

In the amino group substituted with an alkyl group; an aryl group; or a heterocyclic group, the substituents may further have one or more substituents. This amino group is not an unsubstituted amino group. The alkylamino group preferably is an alkylamino group having 1 to 6 carbon atoms. Examples of the substituents include ionic hydrophilic groups. Examples of the alkylamino group include a methylamino group and a diethyl amino group. The aryl amino group may be an arylamino group having one or more substituents or an unsubstituted arylamino group. The aryl amino group preferably is an arylamino group having 6 to 12 carbon atoms. Examples of the substituents include halogen atoms and ionic hydrophilic groups. Examples of the arylamino group include anilino and 2-chloroanilino.

The acylamino group may be an acylamino group having one or more substituents. The acylamino group preferably is an acylamino group having 2 to 12 carbon atoms. Examples of the substituents include ionic hydrophilic groups. Examples of the acylamino group include acetylamino, propionylamino, benzoylamino, N-phenylacetylamino, and 3,5-disulfobenzoylamino.

The ureido group may be a ureido group having one or more substituents or unsubstituted ureido. The ureido group preferably is a ureido group having 1 to 12 carbon atoms. Examples of the substituents include alkyl groups and aryl groups. Examples of the ureido group include 3-methylureido, 3,3-dimethylureido, and 3-phenylureido.

The sulfamoylamino group may be a sulfamoylamino group having one or more substituents or unsubstituted sulfamoylamino. Examples of the substituents include alkyl groups. Examples of the sulfamoylamino group include N,N-dipropylsulfamoylamino.

The alkoxycarbonylamino group may be an alkoxycarbonylamino group having one or more substituents or an unsubstituted alkoxycarbonylamino group. The alkoxycarbonylamino group preferably is an alkoxycarbonylamino group having 2 to 12 carbon atoms. Examples of the substituents include ionic hydrophilic groups. Examples of the alkoxycarbonylamino group include ethoxycarbonylamino.

The aryloxycarbonylamino group may be an aryloxycarbonylamino group having one or more substituents or an unsubstituted aryloxycarbonylamino group. The aryloxycarbonylamino group preferably is an aryloxycarbonylamino group having 7 to 12 carbon atoms. Examples of the substituents include ionic hydrophilic groups. Examples of the aryloxycarbonylamino group include phenoxycarbonylamino.

The alkyl- and aryl-sulfonylamino groups may be alkyl- and aryl-sulfonylamino groups having one or more substituents or unsubstituted alkyl- and aryl-sulfonylamino groups. The sulfonylamino groups preferably are sulfonylamino groups having 1 to 12 carbon atoms. Examples of the substituents include ionic hydrophilic groups. Examples of the sulfonylamino groups include methanesulfonylamino, N-phenylmethanesulfonylamino, benzenesulfonylamino, and 3-carboxybenzenesulfonylamino.

The alkyl-, aryl-, and heterocycle-thio groups may be alkyl-, aryl-, and heterocycle-thio groups having one or more substituents or unsubstituted alkyl-, aryl-, and heterocycle-thio groups. The alkyl-, aryl-, and heterocycle-thio groups preferably are ones having 1 to 12 carbon atoms. Examples of the substituents include ionic hydrophilic groups. Examples of the alkyl-, aryl-, and heterocycle-thio groups include methylthio, phenylthio, and 2-pyridylthio.

Examples of the alkyl- and aryl-sulfonyl groups include methanesulfonyl and phenylsulfonyl, respectively.

Examples of the alkyl- and aryl-sulfinyl groups include methanesulfinyl and phenylsulfinyl, respectively.

The sulfamoyl groups may be a sulfamoyl group having one or more substituents or unsubstituted sulfamoyl. Examples of the substituents include alkyl groups. Examples of the sulfamoyl group include dimethylsulfamoyl and di(2-hydroxyethyl)sulfamoyl.

Specific examples of the azo dye represented by general formula (1) are shown below, but the azo dye to be used in the invention should not be construed as being limited to the following examples. The ionic hydrophilic groups which may be possessed by the dye of the invention may be carboxyl, phosphono, and sulfo groups (including these groups in a salt state). Examples of the counter ion as a component of the salt include an ammonium ion, alkali metal ions (e.g., lithium ion, sodium ion, and potassium ion), and organic cations (e.g., tetramethylammonium ion, tetramethylguanidinium ion, and tetramethylphosphonium ion).

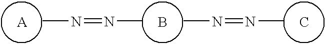

| | A | B | C |
|---|---|---|---|
| 1-1 | 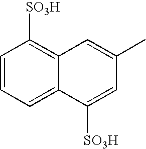 | 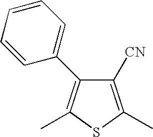 | 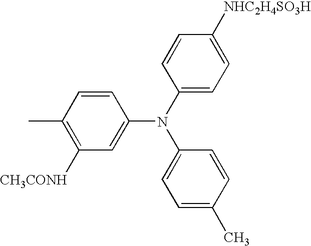 |
| 1-2 | 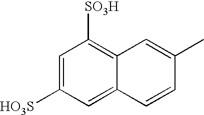 | 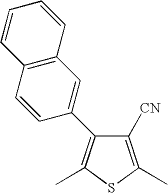 | 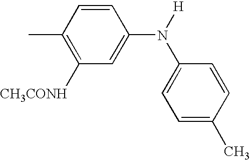 |
| 1-3 | 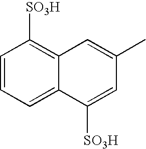 | 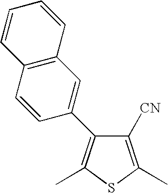 | 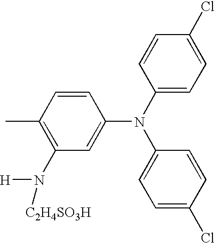 |
| 1-4 | 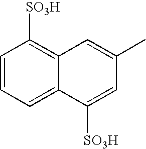 | 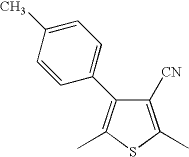 | 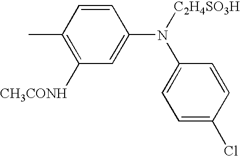 |
| 1-5 | 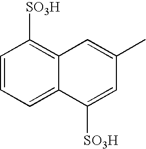 | 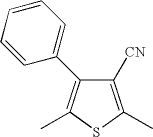 | 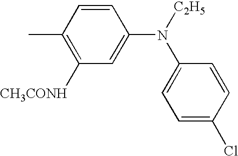 |
| 2-1 | 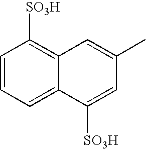 | 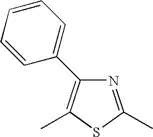 | 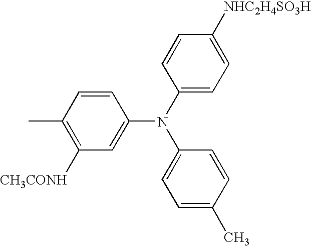 |
| 2-2 | 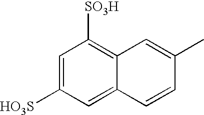 | 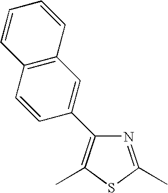 | 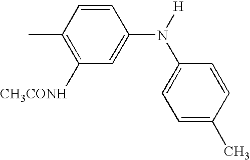 |
| 2-3 | 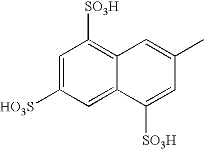 | 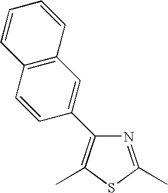 | 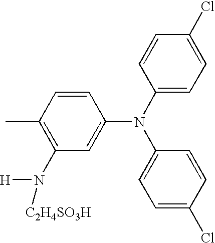 |
| 2-4 | 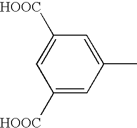 | 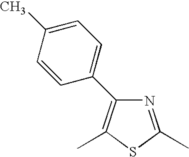 | 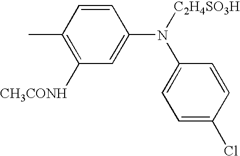 |
| 2-5 | 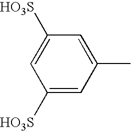 | 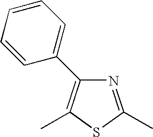 | 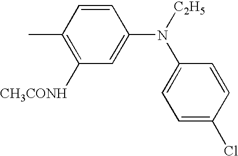 |
| 3-1 | 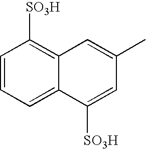 | 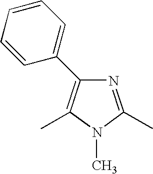 | 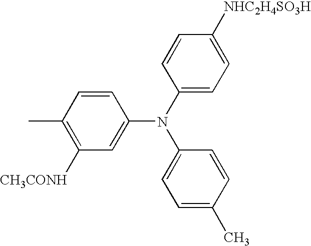 |
| 3-2 | 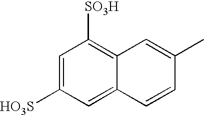 | 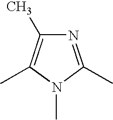 | 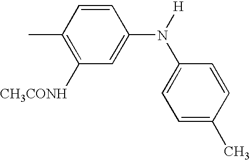 |
| 3-3 | 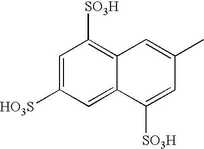 | 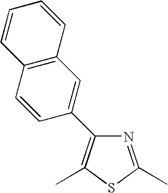 | 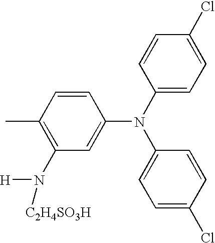 |
| 3-4 | 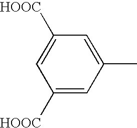 | 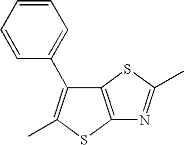 | 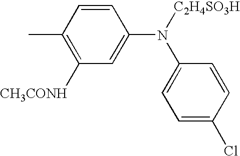 |
| 3-5 | 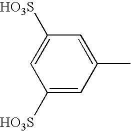 | 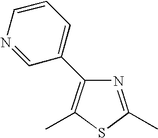 | 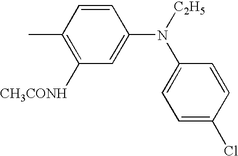 |
| 4-1 | 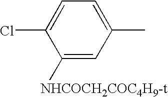 | 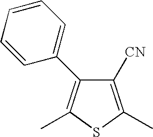 | 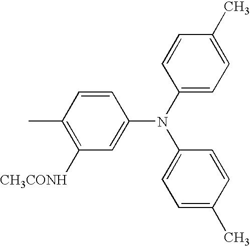 |
| 4-2 | 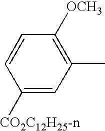 | 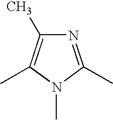 | 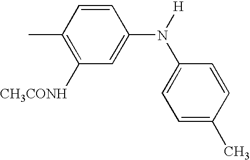 |
| 4-3 | 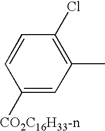 | 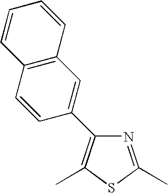 | 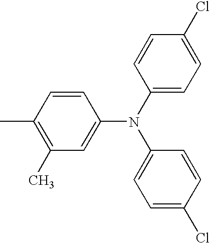 |
| 4-4 | 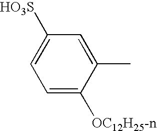 | 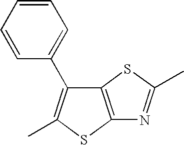 | 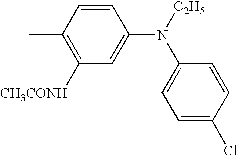 |
| 4-5 | 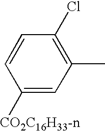 | 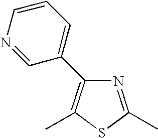 | 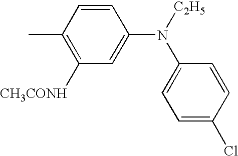 |
| 5-1 | 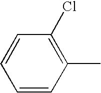 | 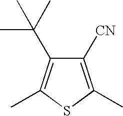 | 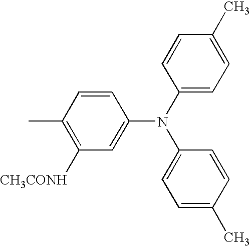 |
| 5-2 | 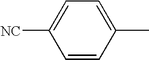 | 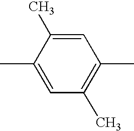 | 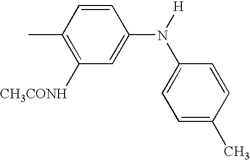 |
| 5-3 | 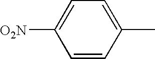 | 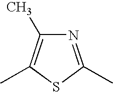 | 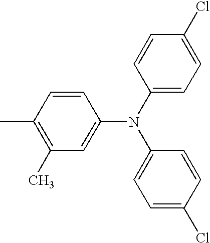 |
| 5-4 | 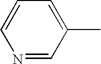 | 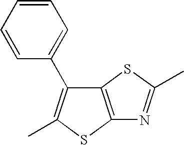 | 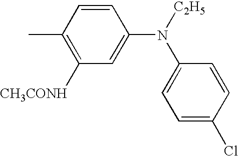 |
| 5-5 | 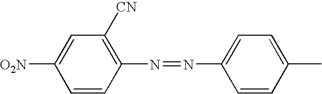 | 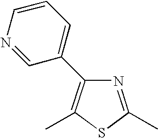 | 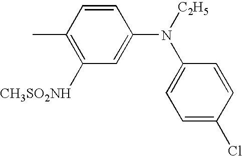 |

With respect to the production of azo dyes represented by general formula (1), Dye (1-1) Synthesis Example will be given later as Example 1. The other dyes can be synthesized in the same manner.

The dye of the invention to be used is regulated, by selecting substituents, so that properties such as solubility, dispersibility, and thermal transferability become suitable for the intended use thereof. Furthermore, the dye of the invention can be used in the state of being dissolved or being emulsified/dispersed or even in a solid dispersion state according to the system in which the dye is to be used.

Ink

The ink (preferably ink for ink-jet recording) of the invention can be produced by dissolving and/or dispersing the azo dye in an oleophilic medium or aqueous medium. Preferred is the case where an aqueous medium is used. Other additives are incorporated according to need in such a degree as not to impair the effects of the invention. Examples of the optionally usable additives include known additives such as drying inhibitors (wetting agents), fading inhibitors, emulsion stabilizers, penetrating agents, ultraviolet absorbers, antiseptics, fungicides, pH regulators, surface tension modifiers, antifoamers, viscosity modifiers, dispersants, dispersion stabilizers, rust preventives, and chelating agents (such additives are shown in, e.g., JP-A-2003-306623). In the case of an ink in an aqueous solution form, such various additives are directly added to the ink. In the case where an oil-soluble dye is used in the form of a dispersion, the general method is to add the additives to the dye dispersion prepared. However, it is also possible to add the additives to an oil phase or an aqueous phase during the preparation of the dispersion.

In the ink for ink-jet recording of the invention, the azo dye may be used in combination with one or more other dyes and/or pigments (shown in, e.g., JP-A-2003-306623) for the purpose of color tone regulation for obtaining full-color images.

There are no limitations on the method of ink-jet recording in the invention, and the ink can be used in ink-jet recording conducted by known techniques such as, e.g., the charge control method in which electrostatic attracting force is used to eject an ink, the drop-on-demand method (pressure pulse method) in which the vibratory pressure caused by a piezoelectric element is utilized, the acoustic ink-jet method in which electrical signals are converted to an acoustic beam and the beam is caused to strike on an ink to eject the ink by means of the emission pressure, and the thermal ink-jet (bubble jet) method in which an ink is heated to generate bubbles and the resultant pressure is utilized.

Techniques for ink-jet recording include: a method in which a low-concentration ink called a photoink is employed and many droplets of the ink which each have a small volume are ejected; a method in which two or more inks which have substantially the same hue and differ in density are used to improve image quality; and a method in which a colorless transparent ink is used. The volume of each ink droplet is controlled mainly by modifying the printing head.

For example, in the case of thermal ink-jet recording, the volume of each droplet can be controlled by modifying the structure of the printing head. Namely, droplets having a desired size can be ejected by changing the sizes of the ink chamber, heating part, and nozzle. In the case of thermal ink-jet recording also, it is possible to realize the ejection of droplets having two or more sizes by mounting two or more printing heads differing in the size of the heating part or nozzle.

In the case of the drop-on-demand recording employing a piezoelectric element, it is possible to change the volume of each droplet by modifying the structure of the printing head as in the thermal ink-jet printing. However, printing heads having the same structure can be made to eject droplets having two or more sizes by regulating the waveform of driving signals for driving the piezoelectric element, as will be described later.

Color Toner

For preparing the color toner containing the dye of the invention, all binder resins for general use in toners can be used. Examples thereof include styrene resins, acrylic resins, styrene/acrylic resins, and polyester resins.

Fine inorganic particles or fine organic particles may be externally added to the toner for the purpose of improving flowability or imparting suitability for charge control, etc. It is preferred to use fine silica particles or fine titanium particles whose surface has been treated with, e.g., a coupling agent containing an alkyl group. These particles preferably are ones having a number-average primary-particle diameter of 10 to 500 nm, and are preferably added to the toner in an amount of 0.1 to 20% by weight.

All release agents which have been used for toners can be used. Examples thereof include olefins such as low-molecular polypropylene, low-molecular polyethylene, and ethylene/propylene copolymers, microcrystalline waxes, carnauba wax, Sasol wax, and paraffin wax. It is preferred that these release agents be added to the toner in an amount of 1 to 5% by weight.

A charge control agent may be added according to need. However, a colorless one is preferred from the standpoint of color formation. Examples thereof include ones having a quaternary ammonium salt structure and ones having a calixarene structure.

As a carrier may be used either an uncoated carrier constituted only of particles of a magnetic material, such as iron or a ferrite, or a resin-coated carrier obtained by coating the surface of particles of a magnetic material with, e.g., a resin. The average particle diameter of this carrier is preferably 30 to 150 μm in terms of volume-average particle diameter.

Methods of image formation to which the toner of the invention is applied are not particularly limited. Examples thereof include: a method in which a color image is repeatedly formed on a photoreceptor and then transferred to form an image; and a method in which images formed on photoreceptors are successively transferred to an intermediate transfer member or the like to form a color image on the intermediate transfer member and this image is transferred to an image-forming member, e.g., paper, to form a color image thereon.

Heat-Sensitive Recording Material

The heat-sensitive recording material of the invention is constituted of: an ink sheet obtained by applying the dye of the invention to a substrate together with a binder; and an image-receiving sheet to which the dye which migrates to areas corresponding to the heat energy applied by a thermal head according to image-recording signals is to be fixed. The ink sheet can be formed by dissolving the dye and a binder in a solvent or finely dispersing the dye and a binder in a medium to thereby prepare an ink, applying the ink to a substrate, and suitably drying the ink.

With respect to the binder resin, ink medium, substrate, and image-receiving sheet to be used, ones described in, e.g., JP-A-7-137466 can be advantageously used.

When the heat-sensitive recording material is applied to a heat-sensitive recording material capable of full-color image recording, it is preferred that a black ink sheet containing a black image-forming material comprising at least the dye of the invention, a cyan ink sheet containing a thermally diffusible cyan dye and capable of forming a cyan image, a magenta ink sheet containing a thermally diffusible magenta dye and capable of forming a magenta image, and a yellow ink sheet containing a thermally diffusible yellow dye and capable of forming a yellow image be successively formed on a substrate by coating.

Color Filter

Methods for color filter formation include: a method in which a pattern is first formed with a photoresist and then dyed; and a method in which a pattern is formed from a photoresist containing a colorant, as disclosed in, e.g., JP-A-4-163552, JP-A-4-128703, and JP-A-4-175753. In introducing the dye of the invention into a color filter, any of these methods may be used. However, preferred examples include the method of color filter formation described in JP-A-4-175753 and JP-A-6-35182. This method comprises applying a positive resist composition comprising a thermosetting resin, quinine diazide compound, crosslinking agent, colorant, and solvent to a base, exposing the resultant coating to light through a mask, developing the coating to remove the exposed areas and thereby form a positive resist pattern, wholly exposing the positive resist pattern to light, and then curing the positive resist pattern. Furthermore, it is possible to form a black matrix in an ordinary manner to obtain a color filter based on the RGB primary color system or Y.M.C complementary color system.

With respect to the thermosetting resin, quinine diazide compound, crosslinking agent, and solvent to be used in the color filter production and to the amounts of these ingredients to be used, those shown in the patent documents cited above can be advantageously used.

### EXAMPLES

The invention will be explained below in detail by reference to Examples, but the invention should not be construed as being limited to these Examples in any way.

### Example 1

### Dye (1-1) Synthesis Example

A mixture of 60 mL of phosphoric acid, 30 mL of acetic acid, and 2.19 mL of nitrosylsulfuric acid (40% sulfuric acid solution) was stirred at 0° C. A solution prepared by dissolving 5.26 g (10 mmol) of the monoazo dye (1) in 15 mL of DMF was gradually added dropwise thereto. The resultant mixture was stirred at 0° C. for 1 hour to yield a diazonium salt. While a suspension of 4.45 g (10 mmol) of the coupler (2) in 182 mL of methanol was kept being stirred at 25° C., the diazonium salt was added dropwise thereto at an internal temperature of 25° C. to cause coupling. After completion of the dropwise addition, the mixture was stirred for 1 hour and the internal temperature was elevated to 50° C. Subsequently, 500 mL of isopropyl alcohol was added dropwise thereto and 3 g of lithium chloride was further added. As a result, a dye precipitated. The dye was taken out by filtration, and crude crystals of the dye obtained were desalted and purified with Sephadex LH-20 carrier, manufactured by Pharmacia, using methanol/water=1/1 (v/v) as an eluent. Thus, dye (1-1) having a high purity was obtained in an amount of 2 g. Yield, 20%; FAB-MASS (Neg.)=982.

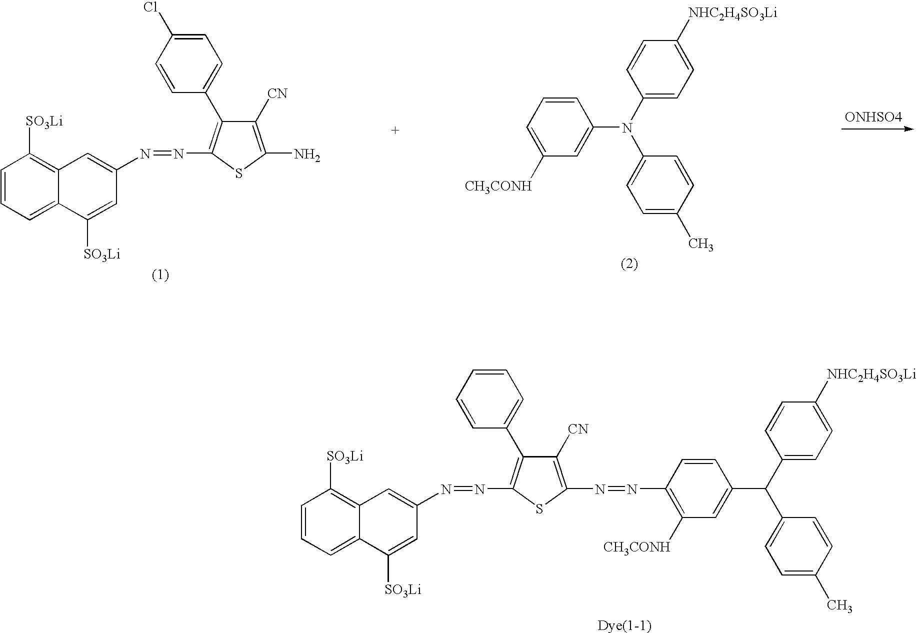

### Example 2

Ultrapure water (resistance, 18 MΩ or higher) and a base were added to the ingredients shown below to adjust the total volume to 1 L. The resultant mixture was stirred for 1 hour with heating at 30 to 40° C. and then filtered under vacuum through a microfilter having an average pore diameter of 0.25 μm. Thus, a black ink Bk-101 was prepared.

Formulation for Black Ink Bk-101

| | |
|---|---|
| Solid Ingredients | |
| Black dye (1—1) of the invention | 60 g/L |
| Proxcel | 5 g/L |
| Urea | 20 g/L |
| Benzotriazole | 3 g/L |
| (Liquid Ingredients) | |
| Diethylene glycol monobutyl ether (DGB) | 100 g/L |
| Glycerol (GR) | 125 g/L |
| Diethylene glycol (DEG) | 100 g/L |
| 2-Pyrrolidone (PRD) | 30 g/L |
| Triethanolamaine (TEA) | 30 g/L |
| Surfynol STG (SW) | 10 g/L |

Subsequently, black inks Bk-102 to Bk-110 were prepared which had the same composition as Bk-101, except that the dye and base in the ink formulation were replaced by those shown in Table 1.

Each ink was charged into a black ink cartridge for ink-jet printer PM-980C, manufactured by EPSON Co., and an image pattern having gray stepwise-changing concentrations was printed.

As the image-receiving sheet for image printing thereon, ink-jet photographic glossy paper “Gasai”, manufactured by Fuji Photo Film Co., Ltd., was used. The inks were evaluated for image quality, ink ejectability, and image fastness.

TABLE 1

| | Dye | Base | Ink pH |
|---|---|---|---|
| Bk-101 | 1—1 | TEA 5 g/L | 8.1 |
| Bk-102 | 1-3 | TEA 5 g/L | 8.1 |
| Bk-103 | 4-3 | TEA 5 g/L | 8.1 |
| Bk-104 | comparative dye 1 | TEA 5 g/L | 8.1 |
| Bk-105 | comparative dye 2 | TEA 5 g/L | 8.1 |
| Bk-106 | comparative dye 3 | TEA 5 g/L | 8.1 |
| Bk-107 | 2—2 | LiOH 2 g/L | 8.2 |
| Bk-108 | comparative dye 3 | LiOH 2 g/L | 8.2 |
| Bk-109 | 4—4 | NaOH 2 g/L | 8.2 |
| Bk-110 | comparative dye 3 | NaOH 2 g/L | 8.2 |

Evaluation Experiment

1) Hue was evaluated by visually examining the broad color tone at around a λₘₐₓ of about 590 nm as the hue of a longer-wavelength black dye. The color tone was evaluated in three ratings, i.e., excellent, good, and poor. The evaluation results obtained are shown in Table 2. In the table given below, A, B, and C show that the hue was excellent, good, and poor, respectively.

2) With respect to image storability for each black dye, gray print samples were subjected to the following evaluations. The image storability was evaluated by examining the density of the gray stepwise pattern with X-rite 310 densitometer equipped with a status A filter. A point having a Dᵥᵢₛ of about 1.0 was used as a sample point, and this point was examined for density change to thereby evaluate image storability.

1. Light fastness was evaluated in the following manner. Immediately after printing, the pattern S was examined for density (Dᵥᵢₛ) Ci. Thereafter, using a weatherometer manufactured by Atlas, the image was irradiated with xenon light (85,000 lx) for 14 days. The density Cf of this pattern S was then measured again and the dye retention, (Cf/Ci)×100, was determined to evaluate the fastness.

The samples in which the dye retention was 80% or higher, those in which the retention was 70 to 80%, and those in which the retention was below 70% are indicated by A, B, and C, respectively.

2. Heat fastness was evaluated by storing each sample for 21 days under the conditions of 80° C. and 70% RH and measuring the density of the pattern S with X-rite 310 before and after the storage to determine the dye retention.

The samples in which the dye retention was 90% or higher, those in which the retention was 80 to 90%, and those in which the retention was below 80% are indicated by A, B, and C, respectively.

3. Ozone resistance (O₃ fastness) was evaluated by allowing each sample to stand for 96 hours in a box having an ozone gas concentration regulated to 5 ppm and measuring the density of the pattern S with X-rite 310 before and after the standing in ozone gas to determine the dye retention.

The ozone gas concentration in the box was regulated with an ozone gas monitor (Model: OZG-EM-01) manufactured by APPLICS.

The samples in which the dye retention was 80% or higher, those in which the retention was 70 to 80%, and those in which the retention was below 70% are indicated by A, B, and C, respectively.

The results obtained are shown in Table 2.

TABLE 2

| | | Light | Heat | O₃ |
|---|---|---|---|---|
| No. | Hue | fastness | fastness | fastness |
| Bk-101 (present | A | A | A | A |
| invention) | | | | |
| Bk-102 (present | A | A | A | A |
| invention) | | | | |
| Bk-103 (present | A | A | A | A |
| invention) | | | | |
| Bk-104 (comparative | B | C | B | C |
| example) | | | | |
| Bk-105 (comparative | B | C | B | C |
| example) | | | | |
| Bk-106 (comparative | B | C | B | C |
| example) | | | | |
| Bk-107 (present | A | A | A | A |
| invention) | | | | |
| Bk-108 (comparative | B | C | B | C |
| example) | | | | |
| Bk-109 (present | A | A | A | A |
| invention) | | | | |
| Bk-110 (comparative | B | C | B | C |
| example) | | | | |

The results given in Table 2 show that the inks prepared according to the formulations which satisfied the requirement in the invention were superior in all performances to the inks of the Comparative Examples.

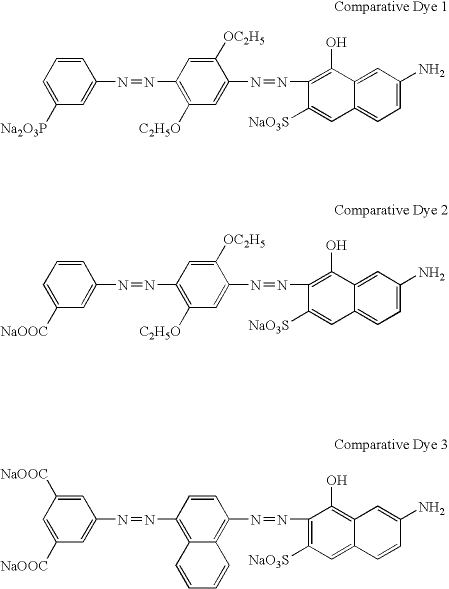

As shown in Table 2, the images (hue) obtained from the inks of the invention (Bk-101, -102, -103, -107, and -109) were better than the images obtained from the comparative inks. In addition, the images obtained from the inks of the invention were excellent in light fastness and resistance to ozone gas.

Furthermore, the inks of the invention (Bk-101, -102, -103, -107, and -109) were used to record an image with an ink-jet printer (PM-700C, manufactured by Seiko Epson Corp.) on glossy paper Super Fine for exclusive use (MJA4S3P, manufactured by Seiko Epson Corp.). The images obtained were evaluated for hue and light fastness. As a result, the same results as given in Table 2 were obtained with respect to each property.

### Example 3

Each of the same inks as those produced in Example 2 was charged into a cartridge for ink-jet printer BJ-F850 (manufactured by Canon Inc.) to print an image with the printer on photographic glossy paper GP-301, manufactured by Cannon Inc. The images obtained were evaluated in the same manners as in Example 2. As a result, the same results as in Example 2 were obtained.

### Example 4

Production of Sample 201

In a mixture of 4.22 g of high-boiling organic solvent (s-2) shown below, 5.63 g of high-boiling organic solvent (s-11) shown below, and 50 mL of ethyl acetate were dissolved 4.83 g of an azo dye (exemplified compound 4-1; oil-soluble dye) and 7.04 g of dioctyl sodium sulfosuccinate at 70° C. To this solution was added 500 mL of deionized water with stirring with a magnetic stirrer. Thus, an oil-in-water type dispersion of coarse particles was produced. This coarse-particle dispersion was passed through Micro-Fluidizer (MICROFLUIDEX INC) five times at a pressure of 600 bar to thereby reduce the particles to finer particles. The resultant emulsion was treated with a rotary evaporator for solvent removal until the emulsion came not to emit the odor of ethyl acetate. Thus, a fine emulsion of the hydrophobic dye was obtained. To this emulsion were added 140 g of diethylene glycol, 50 g of glycerol, 7 g of SURFYNOL 465 (Air Products & Chemicals, Inc.), and 900 mL of deionized water. Thus, an ink was produced.

The high-boiling solvents used have the following structures. 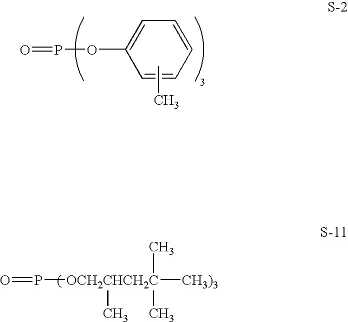
Production of Samples 202 to 204

Samples 202 to 204 were produced in the same manner as for sample 201, except that the azo dye (exemplified compound 4-1; oil-soluble dye) used for sample 201 was replaced by the azo dyes. (oil-soluble dyes) shown in Table 3 given below. The emulsion inks thus obtained as samples 201 to 204 were examined for volume-average particle size with Microtrac UPA (Nikkiso Co., Ltd.). The results obtained are shown in Table 3.

Image Recording and Evaluation

Ink samples 201 to 204 and comparative samples (comparative inks Bk-104 to Bk-106 in Example 2) were subjected to the following evaluations. The results obtained are shown in Table 3.

“Hue”, “Paper dependence”, “Water resistance”, and “Light fastness” in Table 3 are the results of evaluations conducted after image recording with each ink-jet ink on a photographic glossy paper (Ink-Jet Paper, Photo Grade; manufactured by Fuji Photo Film Co., Ltd.) using an ink-jet printer (PM-700C, manufactured by EPSON Co.).

Hue

The image recorded was examined for reflection spectrum at an interval of 10 nm in the 390 to 730 nm region. Furthermore, the broad hue at around a λₘₐₓ of about 590 nm as the hue of a longer-wavelength black dye was visually examined and evaluated in three ratings, i.e., excellent (A), good (B), and poor (C).

Paper Dependence

The image formed on the photographic glossy paper was compared in hue with an image separately formed on plain paper for PPC. Paper dependence was evaluated in two ratings. Namely, the samples in which the difference between the two images was small are indicated by A (good), and those in which the difference between the two images was large are indicated by B (Poor).

Water Resistance

The photographic glossy paper on which an image had been formed was dried at room temperature for 1 hour, subsequently immersed in water for 30 seconds, and then allowed to dry naturally at room temperature. This paper was examined for blurring to evaluate water resistance in three ratings. Namely, the samples which suffered no blurring are indicated by A, those which suffered slight blurring are indicated by B, and those which suffered considerable blurring are indicated by C.

Light Resistance (Light Fastness)

The photographic glossy paper on which an image had been formed was irradiated with xenon light (85,000 lx) with a weatherometer (Atlas C. I65) for 3 days. The density of the image was measured with a reflection densitometer (X-Rite 310TR) before and after the xenon irradiation to evaluate the resistance in terms of dye retention. The reflection density was measured on three points of 1, 1.5, and 2.0.

Light fastness was evaluated in three ratings. Namely, the samples in which the dye retention was 70% or higher at all densities are indicated by A, those in which the dye retention was below 70% at one or two points are indicated by B, and those in which the dye retention was below 70% at all densities are indicated by C.

Ozone Resistance (O₃ Fastness)

The photographic glossy paper on which an image had been formed was allowed to stand for 24 hours in a box having an ozone gas concentration regulated to 0.5 ppm. The density of the image was measured with a reflection densitometer (X-Rite 310TR) before and after the standing in ozone gas to evaluate the resistance in terms of dye retention. The reflection density was measured in three points of 1, 1.5, and 2.0. The ozone gas concentration in the box was regulated with an ozone gas monitor (Model: OZG-EM-01) manufactured by APPLICS.

O₃ fastness was evaluated in three ratings. Namely, the samples in which the dye retention was 70% or higher at all densities are indicated by A, those in which the dye retention was below 70% at one or two points are indicated by B, and those in which the dye retention was below 70% at all densities are indicated by C.

TABLE 3

| | | Average | | | | | |
|---|---|---|---|---|---|---|---|
| | | particle | | Paper | Light | Water | O₃ |
| No. | Dye | size | Hue | dependence | Fastness | resistance | fastness |
| 201 (present | 4-1 | 45 | A | A | A | A | A |
| invention) | | | | | | | |
| 202 (present | 4-2 | 50 | A | A | A | A | A |
| invention) | | | | | | | |
| 203 (present | 4-3 | 48 | A | A | A | A | A |
| invention) | | | | | | | |
| 204 (present | 4—4 | 51 | A | A | A | A | A |
| invention) | | | | | | | |
| Bk-104 | Comparative | — | B | B | C | B | C |
| (comparative | dye 1 | | | | | | |
| example) | | | | | | | |
| Bk-105 | Comparative | — | B | B | C | B | C |
| (comparative | dye 2 | | | | | | |
| example) | | | | | | | |
| Bk-106 | Comparative | — | B | B | C | B | C |
| (comparative | dye 3 | | | | | | |
| example) | | | | | | | |

As apparent from Table 3, the ink-jet inks of the invention had an excellent hue and a small paper dependence and were excellent in water resistance, ozone gas resistance, and light resistance.

### Example 5

Each of the same inks as those produced in Example 4 was charged into a cartridge for ink-jet printer BJ-F850 (manufactured by Canon Inc.) to print an image with the printer on photographic glossy paper GP-301, manufactured by Cannon Inc. The images obtained were evaluated in the same manners as in Example 4. As a result, the same results as in Example 4 were obtained.

The novel azo dye of the invention is a colorant for black which has absorption characteristics (e.g., a broad hue) with excellent color reproduction and further has sufficient fastness to light, heat, moisture, and active gases present in the environment. This novel colorant can hence give colored images and colored materials excellent in hue and fastness. The azo dye can be advantageously used especially for the preparation of an ink for printing, e.g., ink-jet printing, ink sheet for use in heat-sensitive recording materials, color toner for electrophotography, color filter for use in displays such as LCDs and PDPs or photographing elements such as CCDs, dyeing solution for dyeing various fibers, etc.

The ink for ink-jet recording and method of ink-jet recording of the invention, which comprise or use the colored composition described above, can form images having a satisfactory hue and high fastness to light and active gases present in the environment, in particular ozone gas.

The color toner of the invention, which is formed from the colored composition described above, has excellent light fastness, faithful color reproduction, and high OHP quality.

The entire disclosure of each and every foreign patent application from which the benefit of foreign priority has been claimed in the present application is incorporated herein by reference, as if fully set forth.

1. An azo dye represented by formula (1): 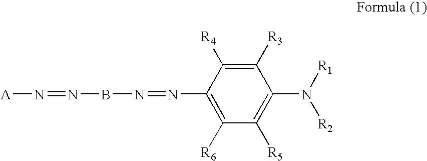
wherein A and B each independently represents an aryl group or a heterocyclic group;
R₁ represents a substituted or unsubstituted aryl group; or a substituted or unsubstituted heterocyclic group;
R₂ represents a hydrogen atom; a substituted or unsubstituted alkyl group; a substituted or unsubstituted aryl group; a substituted or unsubstituted cycloalkyl group; a substituted or unsubstituted alkenyl group; a substituted or unsubstituted aralkyl group; a substituted or unsubstituted heterocyclic group; a substituted or unsubstituted acyl group; an alkyl-substituted sulfonyl group; or an aryl-substituted sulfonyl group;
R₃, R₄, R₅ and R₆ each independently represents a hydrogen atom; a halogen atom; an aliphatic group; an aromatic group; a heterocyclic group; a cyano group; a carboxyl group; a carbamoyl group; an alkoxycarbonyl group; an aryloxycarbonyl group; a heterocycle-oxycarbonyl group; an acyl group; a hydroxy group; an alkoxy group; an aryloxy group; a heterocycle-oxy group; a silyloxy group; an acyloxy group; a carbamoyloxy group; an alkoxycarbonyloxy group; an aryloxycarbonyloxy group; an amino group substituted with an alkyl group, an aryl group or a heterocyclic group; an acylamino group; an ureido group; a sulfamoylamino group; an alkoxycarbonylamino group; an aryloxycarbonylamino group; an alkylsulfonylamino group; an arylsulfonylamino group; a heterocycle-sulfonylamino group; a nitro group; an alkylthio group; an arylthio group; a heterocycle-thio group; an alkylsulfonyl group; an arylsulfonyl group; a heterocycle-sulfonyl group; an alkylsulfinyl group; an arylsulfinyl group; a heterocycle-sulfinyl group; a sulfamoyl group; or a sulfo group;
A, B, R₁, R₂, R₃, R₄, R₅ and R₆ may be substituted; and
R₁ and R₂ may be bonded to R₃ and R₅, respectively, to form a ring.

2. The azo dye according to claim 1,
wherein A is a benzene ring which may be substituted, or a naphthalene ring which may be substituted; and B is a benzene ring which may be substituted, a naphthalene ring which may be substituted, or a heterocycle selected from (a), (b), (c), (d) or (e): 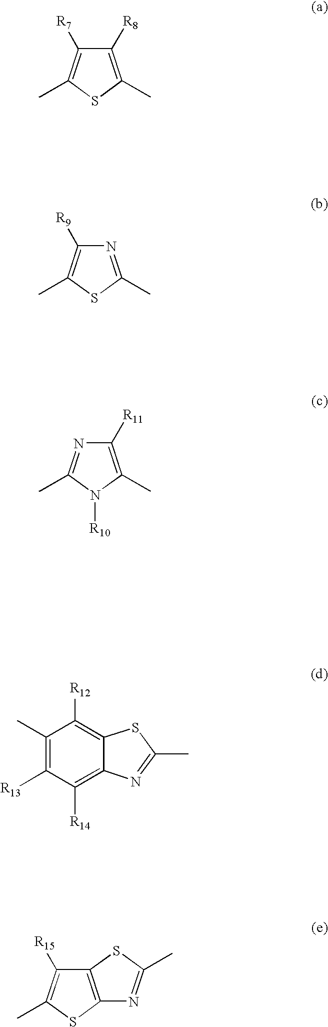
wherein R₇ to R₁₅ each independently represents a hydrogen atom; a halogen atom; an aliphatic group; an aromatic group; a heterocyclic group; a cyano group; a carboxyl group; a carbamoyl group; an alkoxycarbonyl group; an aryloxycarbonyl group; a heterocycle-oxycarbonyl group; an acyl group; a hydroxy group; an alkoxy group; an aryloxy group; a heterocycle-oxy group; a silyloxy group; an acyloxy group; a carbamoyloxy group; an alkoxycarbonyloxy group; an aryloxycarbonyloxy group; an amino group substituted with an alkyl group, an aryl group or a heterocyclic group; an acylamino group; an ureido group; a sulfamoylamino group; an alkoxycarbonylamino group; an aryloxycarbonylamino group; an alkylsulfonylamino group; an arylsulfonylamino group; a heterocycle-sulfonylamino group; a nitro group; an alkylthio group; an arylthio group; a heterocycle-thio group; an alkylsulfonyl group; an arylsulfonyl group; a heterocycle-sulfonyl group; an alkylsulfinyl group; an arylsulfinyl group; a heterocycle-sulfinyl group; a sulfamoyl group; or a sulfo group; and
R₇ to R₁₅ may be substituted.

3. A colored composition for image formation comprising at least one azo dye represented by formula (1) in claim 1.

4. An ink comprising at least one azo dye represented by formula (1) in claim 1.

5. A method of ink-jet recording comprising forming an image with an ink according to claim 4.

6. A heat-sensitive recording material comprising an azo dye according to claim 1.

7. A color toner comprising an azo dye according to claim 1.

8. A color filter comprising an azo dye according to claim 1.
